(19) United States
(12) Patent Application Publication
(10) Pub. No.: US 2024/0382729 A1
(43) Pub. Date: Nov. 21, 2024

(54) MICRONEEDLE DEVICE AND MANUFACTURING METHOD THEREOF

(71) Applicant: DARWIN PRECISIONS CORPORATION, Hsinchu County (TW)

(72) Inventor: YunPei Yang, Hsinchu County (TW)

(21) Appl. No.: 18/591,001

(22) Filed: Feb. 29, 2024

(30) Foreign Application Priority Data
May 19, 2023 (TW) 112118696

(51) Int. Cl. A61M 37/00

(52) U.S. Cl. CPC A61M 37/0015; A61M 2037/0023; A61M 2037/0053

(57) ABSTRACT

A microneedle device includes a substrate and multiple microneedles. The substrate has a first surface. The multiple microneedles are arranged on the first surface. Each of the microneedles includes a conical projection and a shell. The conical projection has a base and a first top opposite to each other. The base is adjacent to the first surface. A material of the conical projection includes a conductive gel. The shell has a second top covering the first top. A material of the shell includes at least one soluble polymer. There is a first distance of 3 μm to 100 μm between the first top and the second top. The microneedle device has the advantages of good conductivity and good needle tip appearance. A manufacturing method of a microneedle device is further provided.

### CROSS-REFERENCE TO RELATED APPLICATION

This application claims the priority benefit of Taiwan Application No. 112118696, filed on May 19, 2023. The entirety of the above-mentioned patent application is hereby incorporated by reference herein and made a part of this specification.

### FIELD OF THE INVENTION

The present invention relates to a microneedle device and a manufacturing method thereof.

### BACKGROUND OF THE INVENTION

Common drug delivery methods include oral administration, subcutaneous injection and transdermal delivery. The transdermal delivery is a method of delivering drugs into the blood circulation system through skin. Therefore, compared with the oral administration and the subcutaneous injection, the transdermal delivery can make the drug concentration in blood stable and avoid pain and wound infection in the case of injection. In recent years, a variety of methods, such as the use of improved chemical molecules, current stimulation, mechanical stimulation and microneedles, have been developed to achieve the purpose of transdermal drug delivery.

The effect of drug delivery by current stimulation is affected by many factors, such as current density, resistance of body surface tissue, drug concentration and molecular weight, etc. The resistance may be reduced by applying a conductive adhesive on the skin or using conductive microneedles. However, if the conductive adhesive is used for a long time, it is easy to cause skin allergy or ulceration. If conductive microneedles made of a conventional hard material are used, the conductive microneedles may break. Therefore, conductive microneedles made of hydrogels can avoid the problems in the above two cases. However, these conductive microneedles may not be formed successfully just because they are elastic and viscous. Nevertheless, if the strength of the conductive microneedles is increased, the conductive microneedles may be formed successfully, but their conductivity will become lower.

### SUMMARY OF THE INVENTION

The present invention provides a microneedle device, which has the advantages of good conductivity and good needle tip appearance.

The present invention further provides a manufacturing method of a microneedle device, which can manufacture a microneedle device with good conductivity and good needle tip appearance.

The microneedle device provided in the present invention includes a substrate and multiple microneedles. The substrate has a first surface, and the multiple microneedles are arranged on the first surface. Each of the microneedles includes a conical projection and a shell. The conical projection has a base and a first top opposite to each other. The base is adjacent to the first surface. A material of the conical projection includes a conductive gel. The shell has a second top covering the first top. A material of the shell includes at least one soluble polymer. There is a first distance between the first top and the second top. The first distance is 3 μm to 100 μm.

In an embodiment of the present invention, the shell further extends to cover the base and the first surface.

In an embodiment of the present invention, the at least one soluble polymer includes at least one of maltose, sucrose, lactose, trehalose, maltodextrin, cyclodextrin, polytriglucose, carboxymethyl cellulose, methyl cellulose, hydroxyethyl cellulose, hydroxymethyl propyl cellulose, polylactic acid, sodium alginate, hyaluronic acid and chitosan.

In an embodiment of the present invention, there is a second distance between the first top and the first surface. The second distance is greater than 30 μm.

In an embodiment of the present invention, a sum of the first distance and the second distance is greater than 150 μm.

In an embodiment of the present invention, the second top has a diameter. The diameter is smaller than 50 μm.

In an embodiment of the present invention, the first top has a diameter. The diameter of the first top is greater than the diameter of the second top.

In an embodiment of the present invention, the microneedle device further includes a current supply component. The substrate has a second surface opposite to the first surface, and the current supply component is connected with the second surface.

In an embodiment of the present invention, the microneedle device further includes a drug. The drug is applied to a side of the shell opposite to the conical projection, arranged in the conical projection, arranged in the shell, or a combination thereof.

The manufacturing method of a microneedle device provided in the present invention includes: pouring a soluble polymer aqueous solution into multiple hollows of a microneedle mold, the soluble polymer aqueous solution including at least one soluble polymer; drying the soluble polymer aqueous solution to form multiple shells, each of the shells having a conical recess; spreading a conductive gel in the conical recesses of the shells, the conductive gel further overflowing from the conical recesses to form a substrate, and the substrate being connected with the conductive gel in the conical recesses; curing the conductive gel; and separating the cured conductive gel and the shells from the microneedle mold.

In an embodiment of the present invention, when the soluble polymer aqueous solution is poured into the hollows, the soluble polymer aqueous solution overflows from the hollows, and when the soluble polymer aqueous solution is dried to form the shells, a connecting portion connected between the shells is further formed.

In an embodiment of the present invention, a weight percentage of the at least one soluble polymer in the soluble polymer aqueous solution is 1.5% to 20%.

In an embodiment of the present invention, before the soluble polymer aqueous solution is dried to form the shells, the method further includes removing bubbles in the soluble polymer aqueous solution by ultrasonic waves, vacuum or centrifugation.

In an embodiment of the present invention, the soluble polymer aqueous solution is dried to form the shells at a temperature of 25 to 60° C.

The microneedle device and the manufacturing method thereof provided in the embodiments of the present invention have the shells made of the soluble polymer, which is conducive to good needle tip appearance of the microneedles, and can maintain the conductivity of the conductive gel.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

The present invention will become more readily apparent to those ordinarily skilled in the art after reviewing the following detailed description and accompanying drawings, in which:

FIG. 1A to FIG. 1E are schematic flowcharts of a manufacturing method of a microneedle device according to an embodiment of the present invention;

FIG. 2 is a partially enlarged cross-sectional schematic diagram of FIG. 1E;

FIG. 3 is a schematic partial three-dimensional view of a microneedle device according to an embodiment of the present invention;

FIG. 4 is a schematic cross-sectional view of a microneedle device according to another embodiment of the present invention; and

FIG. 5 is a schematic cross-sectional view of a microneedle device according to another embodiment of the present invention.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

The above and other technical contents, features and effects of the present invention will be clearly presented in the detailed description of a preferred embodiment below in conjunction with the reference drawings. The directional term mentioned in the embodiment below, such as up, down, left, right, front, or back, is only the direction with reference to the accompanying drawings. Therefore, the directional terms used are used to explain rather than to limit the present invention.

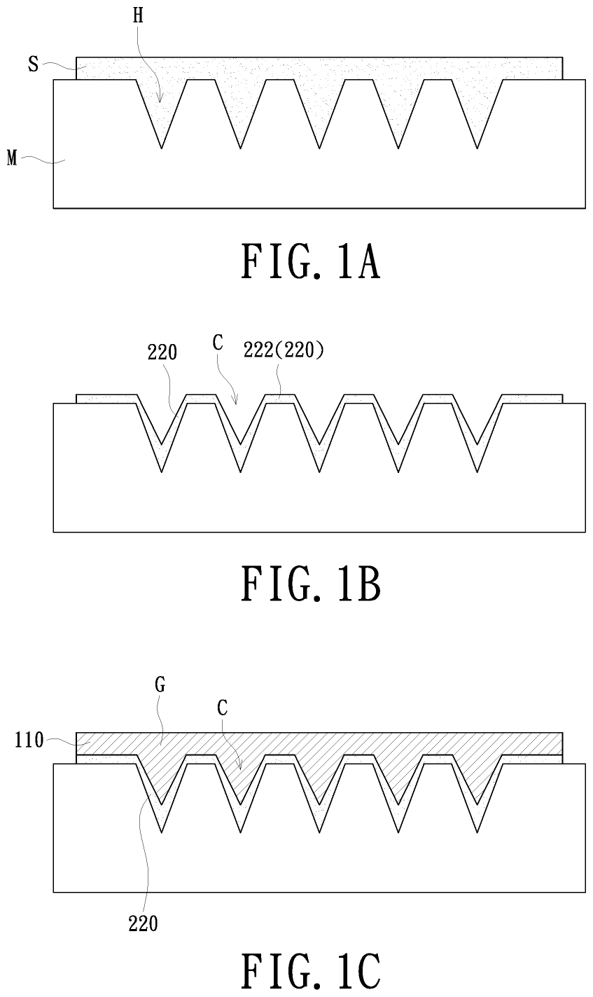

FIG. 1A to FIG. 1E are schematic flowcharts of a manufacturing method of a microneedle device according to an embodiment of the present invention. First, referring to FIG. 1A, the manufacturing method of a microneedle device in this embodiment includes: pouring a soluble polymer aqueous solution S into multiple hollows H of a microneedle mold M. The soluble polymer aqueous solution S includes at least one soluble polymer. Specifically, the at least one soluble polymer may include at least one of maltose, sucrose, lactose, trehalose, maltodextrin, cyclodextrin, polytriglucose, carboxymethyl cellulose, methyl cellulose, hydroxyethyl cellulose, hydroxymethyl propyl cellulose, polylactic acid, sodium alginate, hyaluronic acid and chitosan, but the present invention is not limited thereto. In another embodiment of the present invention, a material that can be decomposed and absorbed through the skin after contacting a human body surface may also be used.

Next, referring to FIG. 1B, the soluble polymer aqueous solution S is dried to form multiple shells 220. Each shell 220 has a conical recess C. Specifically, the soluble polymer aqueous solution S is formed by dissolving the soluble polymer in water. After the soluble polymer aqueous solution S is dried, that is, after the water evaporates, the soluble polymer remains. Therefore, after being dried, the soluble polymer aqueous solution S shrinks in volume to form the shells 220 along the hollows H of the microneedle mold M (as shown in FIG. 1A). Since a shape of the hollow H corresponds to that of the microneedle (conical), a shape of the shell 220 formed along the hollow H also corresponds to that of the microneedle, that is, the shell 220 has the conical recess C. In this embodiment, when the soluble polymer aqueous solution S is poured into the multiple hollows H, the soluble polymer aqueous solution S, for example, overflows from the multiple hollows H. When the soluble polymer aqueous solution S is dried to form the multiple shells 220, a connecting portion 222 connected between the multiple shells 220, for example, is further formed. For example, the soluble polymer aqueous solution S may overflow from the hollows H when being poured, so that the multiple hollows H can be connected by the soluble polymer aqueous solution S. In this way, the surface of the microneedle mold M may be covered with the soluble polymer aqueous solution S, which, after being dried, can form a layer of the shells 220 having the connecting portion 222 on the surface of the microneedle mold M. However, this is not specifically limited in the present invention. In another embodiment, when the soluble polymer aqueous solution S is poured, the soluble polymer aqueous solution S, for example, does not overflow from the hollows H. Besides, in this embodiment, the soluble polymer aqueous solution S is dried to form the multiple shells 220 at a temperature of, for example, 25° C. to 60° C., which, however, is not specifically limited in the present invention.

On the other hand, before the soluble polymer aqueous solution S is dried to form the multiple shells 220, the manufacturing method of a microneedle device 100 may further include: removing bubbles (not shown) in the soluble polymer aqueous solution S by ultrasonic waves, vacuum or centrifugation. Specifically, since the soluble polymer aqueous solution S has a lower fluidity, when the soluble polymer aqueous solution S is poured into the hollows H, the soluble polymer aqueous solution S may not flow easily, thus leaving bubbles in the hollows H to form gaps. If the bubble is just at the tip of the hollow H (i.e., at the top of the shell 220), this may result in a less desirable needle shape at the top of the shell 220 formed after drying. Although this embodiment is disclosed as above, it is not specifically limited.

Next, referring to FIG. 1C, a conductive gel G is spread in the multiple conical recesses C of the multiple shells 220. The conductive gel further overflows from the multiple conical recesses C to form a substrate 110, and the substrate 110 is connected with the conductive gel G in the multiple conical recesses C. Specifically, when the conductive gel G is applied to the conical recesses C, the conical recess C shapes the shape of the conductive gel G, so the conductive gel G is also conical, and the shell 220 covers the conical top of the conductive gel G.

Next, referring to FIG. 1D, the conductive gel G is cured. For example, the curing may be photocuring, that is, the conductive gel G may be cured by irradiation with light L, but the present invention is not limited thereto. In another embodiment, the curing may also be thermal curing. In addition, in this embodiment, before the conductive gel G is cured, the bubbles (not shown) in the conductive gel G may be removed by ultrasonic waves, vacuum or centrifugation. However, since the conductive gel G is covered by the shells 220, even if the bubbles are formed, it will have little effect on the curing of the conductive gel G. Besides, the conductive gel G has better fluidity than the soluble polymer aqueous solution S. Adjustments may be made according to actual needs, which is not specifically limited in the present invention.

Next, referring to FIG. 1E, the cured conductive gel Ga and the multiple shells 220 are separated from the microneedle mold M by, for example, mechanical demolding, such as adsorption with suction cups, which, however, is not specifically limited in the present invention. After the demolding, the microneedle device 100 can be obtained. The microneedle device 100 includes a substrate 110 and multiple microneedles 200. The substrate 110 has a first surface 111. The multiple microneedles 200 are arranged on the first surface 111. Each of the microneedles 200 includes a conical projection 210 and a shell 220. In this embodiment, the conical projections 210 and the substrate 110 are formed, for example, by the cured conductive gel Ga. The conical projection 210 has a first top 211 and a base 212 opposite to each other. The base 212 is adjacent to the first surface 111. The shell 220 has a second top 221 covering the first top 211.

It is worth mentioning that in this embodiment, a thickness and a structural strength of the top of the shell 220 may be adjusted by adjusting a weight percentage of the soluble polymer in the soluble polymer aqueous solution S. Specifically, when the weight percentage of the soluble polymer in the soluble polymer aqueous solution S changes, the thickness of the shell 220 formed after drying also changes accordingly. Besides, the structural strength of the shell 220 also changes according to the type of the soluble polymer. Therefore, the weight percentage may be adjusted according to needs, the type or the formula of the soluble polymer, which is not specifically limited in the present invention. For example, in the manufacturing method of the microneedle device in this embodiment, the weight percentage of the soluble polymer in the soluble polymer aqueous solution S is, for example, 1.5% to 20%. In this case, the structural strength of the shell 220 helps puncture the stratum corneum on the body surface.

Besides, by adjusting the weight percentage of the soluble polymer in the soluble polymer aqueous solution S, the shape of the hollows H, the type of the soluble polymer, or other factors, there may be a first distance L1 between the first top 211 and the second top 221 of the microneedle device 100, and the first distance L1 may be 3 μm to 100 μm. Specifically, the first top 211 of the conical projection 210 is covered by the second top 221 of the shell 220, and the second top 221 of the shell 220 has a thickness of 3 μm to 100 μm, which is beneficial to protecting the first top 211 of the conical projection 210. In terms of the characteristics of the materials, the shell 220 includes the soluble polymer, and the conical projection 210 includes the conductive gel Ga. The conductive gel Ga is viscous and elastic, and the shell 220 is harder and less viscous than the conical projection 210. Therefore, when the conductive gel Ga and the shell 220 are separated from the microneedle mold M (as shown in FIG. 1D), since the shell 220 is connected with the microneedle mold M, the microneedle device 100 can be separated from the microneedle mold M successfully during the demolding. In this way, it helps the microneedle device 100 to form the needle shape, and prevents the conductive gel Ga from directly contacting the microneedle mold M and adhering to the microneedle mold M, resulting in deformation caused by stretching. Besides, the shell 220 has better structural strength than the conical projection 210, which further helps the microneedle device 100 puncture the stratum corneum on the body surface. In other embodiment, the first distance L1 is, for example, 5 μm to 50 μm, 5 μm to 20 μm, or 5 μm to 10 μm, which may be adjusted according use needs and is not specifically limited in the present invention.

The microneedle device 100 of this embodiment uses the shell 220 to cover up the top of the conical projection 210 without adjusting the formula of the conductive gel G, so as to increase the structural strength of the top of the microneedle 200 and improve the appearance of the needle tip of the microneedle 200, so that the microneedle 200 can have the advantages of good conductivity and good needle tip appearance. Therefore, in this embodiment, there may be no need to adjust the formula of the conductive gel G to increase the structural strength of the microneedle 200, which can avoid the reduction in conductivity.

It should be noted that in this embodiment, when the soluble polymer aqueous solution S is poured, for example, to overflow from the hollows H, there is, for example, the connecting portion 222 formed between the multiple shells 220 after drying. In other words, the shell 220 further extends to cover the base 212 and the first surface 111. In another embodiment, when the soluble polymer does not overflow from the hollows H, there is no connecting portion 222 formed between the shells 220 after drying, that is, the shell 220 does not cover the first surface 111 of the substrate 110.

Still referring to FIG. 1E, in this embodiment, there is a second distance L2 between the first top 211 and the first surface 111. The second distance L2 is, for example, greater than 30 μm. In this embodiment, a sum of the first distance L1 and the second distance L2 is, for example, greater than 150 μm. Specifically, when the microneedle device 100 is attached to the body surface, the microneedles 200 puncture the body surface to form microchannels. The stratum corneum located on the outermost layer of the body surface and having a large electric resistance typically has a thickness of 10 μm to 20 μm, so making a length of the conical projection 210 greater than the thickness of the stratum corneum is conducive to current transfer between the microneedle device 100 and the body surface. However, the present invention is not limited thereto, and adjustments may be made according to actual needs.

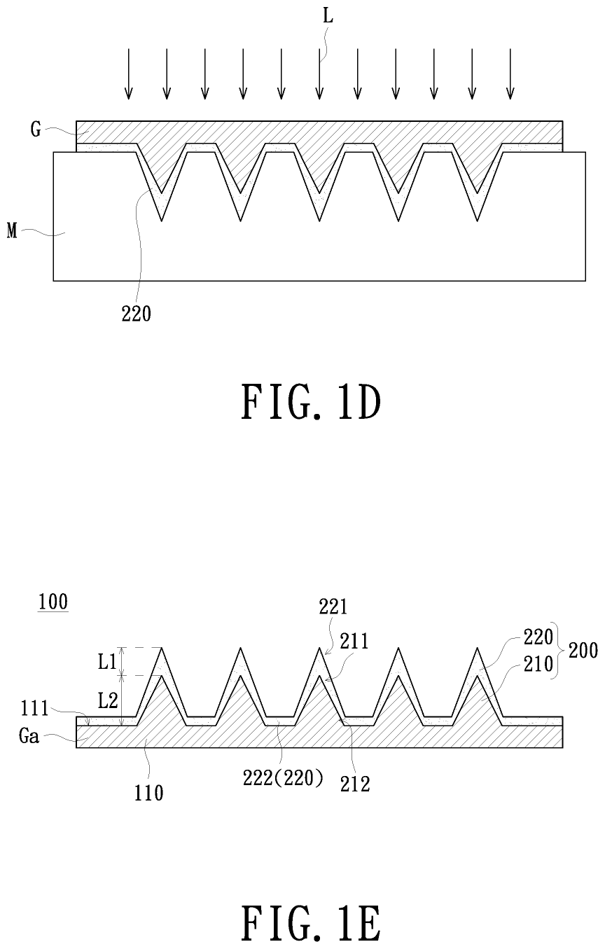

FIG. 2 is a partially enlarged cross-sectional schematic diagram of FIG. 1E. Referring to FIG. 2, in this embodiment, the second top 221 has a diameter D2. The diameter D2 is, for example, smaller than 50 μm. In this case, the second top 221 of the shell 220 has a smaller area, that is, the microneedle 200 has a sharp top, which helps the microneedle device 100 to puncture the body surface, but the present invention is not limited thereto. Besides, in this embodiment, the first top 211 has a diameter D1. The diameter D1 of the first top 211 is, for example, greater than the diameter D2 of the second top 221, but the present invention is not limited thereto.

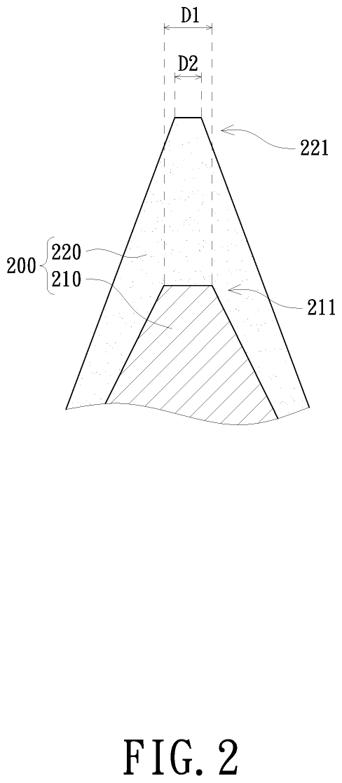

FIG. 3 is a schematic partial three-dimensional view of a microneedle device according to an embodiment of the present invention. Referring to FIG. 3, in this embodiment, the multiple hollows H of the microneedle mold M (as shown in FIG. 1A) may be designed, for example, to be arranged in an array. Each hollow H is in a shape of a triangular pyramid, so that the microneedle device 100 made in accordance with the manufacturing method of the microneedle device has, for example, the multiple microneedles 200 that are orderly arranged in an array. The conical projection 210 of each microneedle 200 is covered with the shell 220. Besides, the microneedle 200 in this embodiment is, for example, in a shape of a triangular pyramid, but this embodiment does not limit the number, arrangement and shape of the hollows H of the microneedle mold M and the microneedles 200.

According to the microneedle device 100 and the manufacturing method thereof provided in the embodiments of the present invention, the shells 220 made of the soluble polymer are conducive to good needle tip appearance of the microneedles 200, and can maintain the conductivity of the conductive gel G.

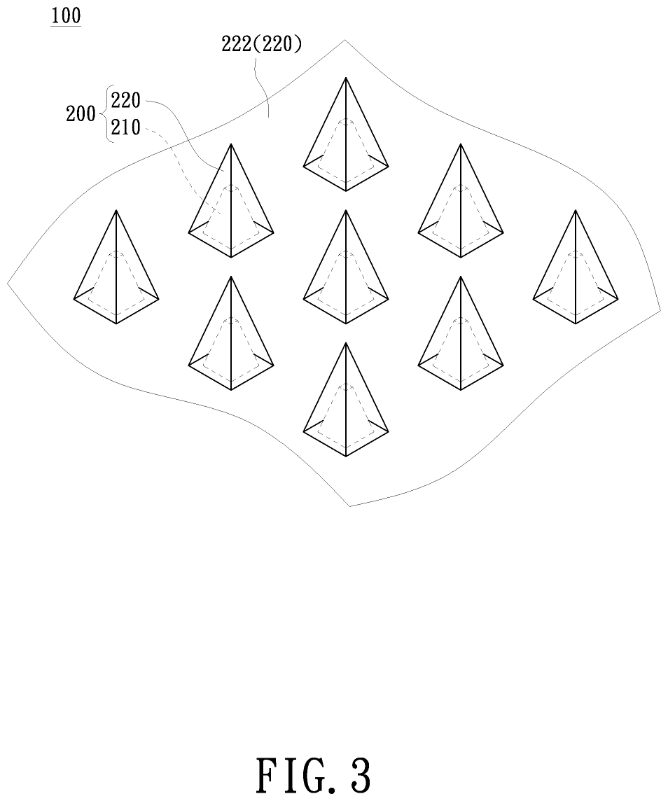

FIG. 4 is a schematic cross-sectional view of a microneedle device according to another embodiment of the present invention. Referring to FIG. 4, the microneedle device 100a of this embodiment is similar to the microneedle device 100 described above, except that the microneedle device 100a further includes, for example, a current supply component 120. The substrate 110 has a second surface 112 opposite to the first surface 111, and the current supply component 120 is connected with the second surface 112. Specifically, the current supply component 120 may provide a current which is transferred to the conical projections 210 formed by the conductive gel and then to the body surface, thereby assisting in transdermal drug delivery. The current supply component 120 includes, for example, an electrode and a power supply, and may also include a thermoelectric material that generates a current by using the temperature difference between human body temperature and room temperature, which, however, is not specifically limited in the present invention.

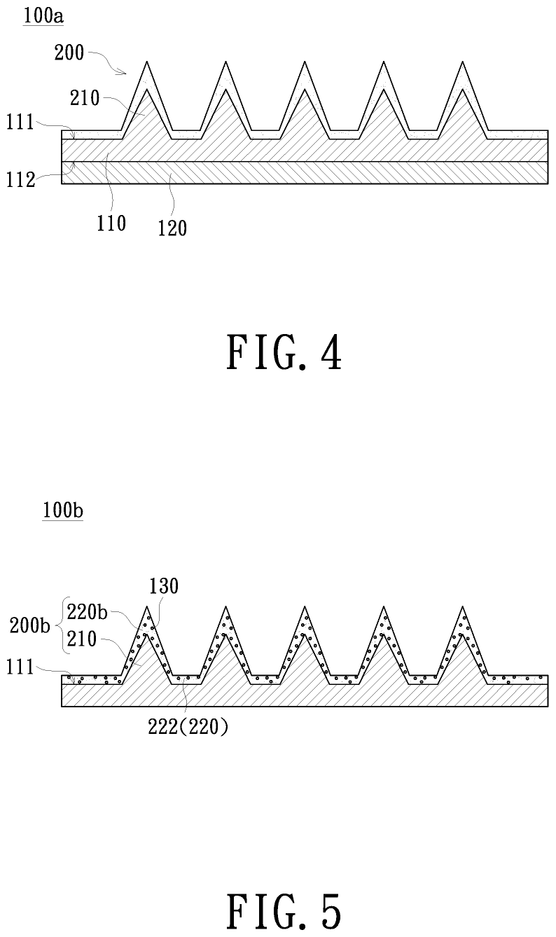

FIG. 5 is a schematic cross-sectional view of a microneedle device according to another embodiment of the present invention. Referring to FIG. 5, the microneedle device 100b of this embodiment is similar to the microneedle device 100 described above, except that the microneedle device 100b further includes a drug 130. The drug 130 is applied to a side of the shell 220b opposite to the conical projection 210, arranged in the conical projection 210, arranged in the shell 220b, or a combination thereof. For example, in this embodiment, the drug 130 is mixed in the soluble polymer aqueous solution S during the manufacture of the microneedle device 100b, so that after the soluble polymer aqueous solution S is dried to form the shells 220b, the drug 130 can be located in the shells 220b, but the present invention is not limited thereto. In another embodiment, the drug 130 may also be mixed with the conductive gel during the manufacture of the microneedle device 100b, so that after the conductive gel is cured, the drug 130 can be located in the conical projections 210. The drug may further be arranged in the substrate 110. In a further embodiment, the drug 130 may also be applied to the side of the shells 220b opposite to the conical projections 210, for example, to the second tops 221, or the second tops 221 and the connecting portions 222, which is not specifically limited in the present invention.

In summary, the microneedle device and the manufacturing method thereof provided in the present invention have the shells made of the soluble polymer, which is conducive to good needle tip appearance of the microneedles, and can maintain the conductivity of the conductive gel.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A microneedle device, comprising:
a substrate, having a first surface; and
multiple microneedles, arranged on the first surface, each of the microneedles comprising:
a conical projection, having a base and a first top opposite to each other, the base being adjacent to the first surface, and a material of the conical projection comprising a conductive gel; and
a shell, having a second top covering the first top, a material of the shell comprising at least one soluble polymer, wherein there is a first distance between the first top and the second top, the first distance being 3 μm to 100 μm.

2. The microneedle device according to claim 1, wherein the shell further extends to cover the base and the first surface.

3. The microneedle device according to claim 1, wherein the at least one soluble polymer comprises at least one of maltose, sucrose, lactose, trehalose, maltodextrin, cyclodextrin, polytriglucose, carboxymethyl cellulose, methyl cellulose, hydroxyethyl cellulose, hydroxymethyl propyl cellulose, polylactic acid, sodium alginate, hyaluronic acid and chitosan.

4. The microneedle device according to claim 1, wherein there is a second distance between the first top and the first surface, the second distance being greater than 30 μm.

5. The microneedle device according to claim 4, wherein a sum of the first distance and the second distance is greater than 150 μm.

6. The microneedle device according to claim 1, wherein the second top has a diameter, the diameter being smaller than 50 μm.

7. The microneedle device according to claim 6, wherein the first top has a diameter, the diameter of the first top being greater than the diameter of the second top.

8. The microneedle device according to claim 1, further comprising a current supply component, wherein the substrate has a second surface opposite to the first surface, and the current supply component is connected with the second surface.

9. The microneedle device according to claim 1, further comprising a drug, wherein the drug is applied to a side of the shell opposite to the conical projection, arranged in the conical projection, arranged in the shell, or a combination thereof.

10. A manufacturing method of a microneedle device, comprising:
pouring a soluble polymer aqueous solution into multiple hollows of a microneedle mold, the soluble polymer aqueous solution comprising at least one soluble polymer;
drying the soluble polymer aqueous solution to form multiple shells, each of the shells having a conical recess;
spreading a conductive gel in the conical recesses of the shells, the conductive gel further overflowing from the conical recesses to form a substrate, and the substrate being connected with the conductive gel in the conical recesses;
curing the conductive gel; and
separating the cured conductive gel and the shells from the microneedle mold.

11. The manufacturing method of a microneedle device according to claim 10, wherein when the soluble polymer aqueous solution is poured into the hollows, the soluble polymer aqueous solution overflows from the hollows, and when the soluble polymer aqueous solution is dried to form the shells, a connecting portion connected between the shells is further formed.

12. The manufacturing method of a microneedle device according to claim 10, wherein the at least one soluble polymer comprises at least one of maltose, sucrose, lactose, trehalose, maltodextrin, cyclodextrin, polytriglucose, carboxymethyl cellulose, methyl cellulose, hydroxyethyl cellulose, hydroxymethyl propyl cellulose, polylactic acid, sodium alginate, hyaluronic acid and chitosan.

13. The manufacturing method of a microneedle device according to claim 10, wherein a weight percentage of the at least one soluble polymer in the soluble polymer aqueous solution is 1.5% to 20%.

14. The manufacturing method of a microneedle device according to claim 10, wherein before the soluble polymer aqueous solution is dried to form the shells, the method further comprises removing bubbles in the soluble polymer aqueous solution by ultrasonic waves, vacuum or centrifugation.

15. The manufacturing method of a microneedle device according to claim 10, wherein the soluble polymer aqueous solution is dried to form the shells at a temperature of 25 to 60° C.
